(19) United States
(12) Patent Application Publication
(10) Pub. No.: US 2026/0124393 A1
(43) Pub. Date: May 7, 2026

(54) DOWN TITRATION LOADING OF SOTALOL INTRAVENOUSLY

(71) Applicant: Academic Pharmaceuticals, INC., Lake Bluff, IL (US)

(72) Inventor: John Charin Somberg, Lake Forest, IL (US)

(21) Appl. No.: 18/939,387

(22) Filed: Nov. 6, 2024

(51) Int. Cl. A61M 5/172; A61K 9/00; A61K 31/145

(52) U.S. Cl. CPC A61M 5/1723; A61K 9/0019; A61K 9/0053; A61K 31/145; A61M 2230/06

(57) ABSTRACT

The present invention relates to a method of loading sotalol intravenously that starts with the highest approved doses, evaluates the QTc effects, and selects the highest does that is represented by a maximum serum drug concentration that does not excessively prolong the QTc interval on the electrocardiogram. The method further relates to down titration if needed.

### BACKGROUND OF THE INVENTION

Sotalol is an anti-arrhythmic agent primarily used to prevent recurrence of highly symptomatic atrial fibrillation or atrial flutter (AF/AFC). Loading of sotalol has been routinely performed by oral drug administration over 3 days of in hospital observation to determine the effect of a given oral dose on the QTc interval. QTc prolongation on the ECG represents prolongation in cardiac repolarization. Prolongation in cardiac repolarization is the primary mechanism by which sotalol prevents AF/AFL recurrence. However, excessive prolongation of ventricular repolarization as seen as prolongation of the QTc interval can precipitate cardiac arrhythmias developing in the ventricular muscle of the heart. These arrhythmias may be ventricular tachycardia (VT) or ventricular fibrillation (VF). They are often proceeded by a fast ventricular arrhythmia termed torsade de pointe VT, that may be life threatening or degenerate into polymorphic VT or VF. Studies have reported that QTc prolongation in excess of 500 msec or 550 msec in patients with conduction abnormalities increases the frequency of these serious arrhythmias. Thus the U.S. FDA has recommended stopping sotalol therapy or reducing the dose of sotalol that would result in a reduced QTc prolongation. To reduce the time in hospital loading, IV administration of sotalol has been utilized to achieve a maximum steady state concentration of a selected dose in a shortened period of time permitting the evaluation of the effects of this concentration on the QTc interval and thus the potential for life threatening arrhythmias. Somberg (U.S. Pat. No. 10,512,620B1) has taught to load sotalol IV to the low target dose of sotalol that is 80 mg and then if tolerated to escalate to higher doses of oral sotalol 120 and 160 mg. This dose escalation initially takes 24 hours. If dose is tolerated (QTc prolongation acceptable) then patients can be brought back to the hospital for further loading with dose escalation initiated again, prolonging the in-hospital loading to 2, 3, or 4 days. What usually occurs is loading to 80 mg and discharge at the lowest and least effective dose of sotalol employed for chronic oral dosing. Given the additional time requirements for up titration to higher doses, the additional titration is often not undertaken. The lack of up titration of sotalol initially leads to patients often receiving inadequate doses of sotalol as maintenance therapy to prevent recurrence of AF. The inevitable recurrence of arrhythmias requiring hospitalization leading to increased dosing or switching to an alternative drug therapy is inconvenient but more serious is the development of an AF/AFL recurrence which can lead to embolization and stroke with severe consequences to the patient.

It would be beneficial to find a way to achieve a higher dose of sotalol without a second titration/hospital stay (up titration). It would also be beneficial to avoid re-hospitalization due to a recurrence of AF/AFL because of inadequate sotalol dosing.

### SUMMARY OF THE INVENTION

Accordingly, in some aspects, there are described methods of intravenous (IV) loading of sotalol starting at the calculated highest tolerated dose and down titrating if QT prolongation is excessive.

In some aspects, there are described novel methods of IV loading of sotalol starting at the calculated highest tolerated dose, followed by oral dosing 4 hours after completion of the IV loading dose.

In some aspects, there are described novel methods of initiating sotalol by IV loading starting at the calculated highest tolerated dose, following by oral dosing, and discharging a patient about 9-17 hours after initiating the IV.

These and other objects, which will become apparent during the following detailed description, have been achieved by the inventors' discovery that the presently claimed methods of starting sotalol titration at the calculated highest tolerated dose and down-titrating where necessary.

### BRIEF DESCRIPTION OF THE DRAWINGS

FIG. 1 shows an example of a pharmacokinetic sequence for an intravenous loading dose followed by first and second oral doses.

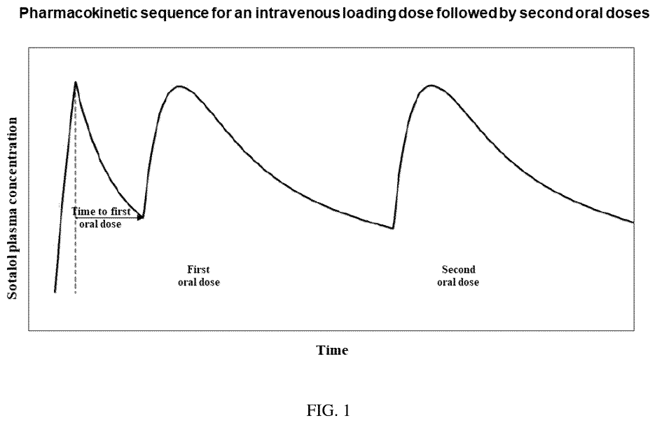

FIG. 2 shows a flow chart for the IV initiation of sotalol at 120 mg and creatinine clearance of >90 mL/min and down titrating if QT prolongation is excessive.

### DETAILED DESCRIPTION OF THE INVENTION

All references cited herein are hereby incorporated in their entirety herein by reference.

### Definitions

a. BID: twice daily;
b. BP: blood pressure;
c. HR: heart rate;
d. IV: intravenous or intravenously;
e. PO: per os, orally;
f. QT interval: time from the start of the Q wave to the end of the T wave in an electrocardiogram; and,
g. QTc: corrected QT interval for heart rate.

### Method of Sotalol IV Loading

The IV loading of sotalol is typically required to be initiated in a facility that can provide continuous ECG monitoring and cardiac resuscitation. Personnel are typically trained in the management of serious life-threatening ventricular arrhythmias. In some instances, withdrawal of other antiarrhythmic therapy is completed before initiating sotalol therapy.

In an aspect, the invention relates to a method of targeting a starting oral dose of 120 mg PO BID sotalol (160 mg in patients weighing >90 kg). This is done by loading sotalol intravenously (IV) over 1 hour with the loading dose selection determined by renal function. If at 1 hour (completion of the IV) the QTc is <500 msec, a first oral dose of sotalol can be given 4 hours after the infusion is complete. The patient can then be discharged after 4 hours if the QTc has not exceeded 500 msec (550 in patients with conduction abnormalities) (˜9 hours from starting the IV dosing). Alternatively, 12 hours after the first oral dose, if the QTc has not exceeded 500 msec (550 in patients with conduction abnormalities) the patient can be given a second oral dose of sotalol and then discharged. The discharged patient is prescribed sotalol BID at the dose that was targeted with the IV infusion. As outlined below, the timing and dose can be modified (e.g., down titrated) based on the patient's creatinine clearance and QTc response to the IV and first oral dose.

One type of patient expected to benefit from one or more of the aspects described herein is the patient who was in highly symptomatic AF/Aflutter, but is now in sinus rhythm, and who is indicated for chronic oral sotalol therapy. Such patients could (a) have not previously received sotalol therapy; or (b) have received it in the past but stopped therapy (are not currently on sotalol therapy).

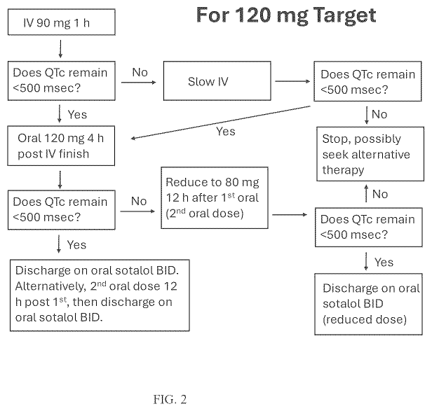

In another aspect, as outlined in FIG. 2, if during the IV the QTc interval is greater than considered safe (e.g., QTc>500 msec), then the IV can be slowed. If the QTc interval is then considered safe (<500 msec) the first oral dose at the selected target (e.g., 120 mg or 160 mg) can be given. If slowing the IV does not remedy the unsafe QTc, then the IV is stopped and alternative therapy is sought. Doses and timing based on creatinine clearance are described in Table A below.

In another aspect, as outlined in FIG. 2, if after receiving the first selected oral dose (e.g., 120 or 160 mg), the QTc interval is greater than considered safe (e.g., >500 msec), then the second oral dose can be reduced (e.g., 80 or 120 mg). If after receiving the second oral dose, the QTc interval is considered safe (<500 msec), the patient can be discharged on sotalol BID at the lower dose. If after receiving the reduced oral dose (second oral dose), the QTc interval is greater than considered safe, then the oral sotalol is stopped and alternative therapy is sought. Doses and timing based on creatinine clearance are described in Table A below.

In another aspect, the QTc of the patient to receive the sotalol titration is determined. For example, a 12-lead ECG can be performed to determine the QTc interval. If the QTc is greater than 450 msec sotalol is not recommended.

In another aspect, the calculated highest target dose is based on the patients calculated creatinine clearance (see table A).

TABLE A
Dosing Parameters

| | | | | |
|---|---|---|---|---|
| | 1 hour IV | Minimum delay | Next oral | Discharge |
| | loading | to first | dosing | (hours |
| CrCL* | dose | oral dose | interval | after IV |
| (mL/min) | (mg)** | (hours)*** | (hours)**** | start) |

Target Dose 120 mg, Patient Weight ≤90 kg

| | | | | |
|---|---|---|---|---|
| >90 | 90 | 4 | 12 | 9-12 h (1 oral dose), |
| | | | | 12-23 h (2 oral doses) |
| >60-90 | 125 | 4 | 12 | 9-12 h (1 oral dose), |
| | | | | 12-23 h (2 oral doses) |
| >30-60 | 112.5 | 6 | 24 | 31-39 |
| 10-30 | 112.5 | 12 | 48 | 61-69 |

Target Dose 160 mg, Patient Weight >90 kg

| | | | | |
|---|---|---|---|---|
| >90 | 120 | 4-8 | 12 | 9-12 h (1 oral dose), |
| | | | | 12-23 h (2 oral doses) |
| >60-90 | 165 | 4-8 | 12 | 9-12 h (1 oral dose), |
| | | | | 12-23 h (2 oral doses) |
| >30-60 | 150 | 6 | 24 | 31-39 |
| 10-30 | 150 | 12 | 48 | 61-69 |

*Creatinine Clearance: calculated using the Cockeroft Gault formula.
**Can be lengthened beyond 1 h if required by prolongated QTc.
***Oral dose to be given presuming the patient's QTc is determined to be acceptable.
****Based on when the first oral dose is given and presumes no down-titration.

In another aspect, the invention relates to a down-titration sequence for loading sotalol, comprising:
a) administering intravenously (IV) to a patient in need thereof, over 1 hour, a loading dose based on the calculated highest oral target dose based on weight, with the IV loading dose being adjusted for renal function as shown in Table A;
b) optionally, slowing the infusion rate if the QTc increases to >500 msec and/or stopping the infusion if there is persistent QTc prolongation >500 msec;
c) if the QTc is <500 msec at 4 hours after completion of the IV loading dose, administering a first oral dose of sotalol based on the highest target oral dose selected for step a), provided that if the patient has a creatinine clearance of ≤60 mL/min then the timing of the first oral dose is delayed as described in Table A:
d) if the QTc is <500 msec at 4 hours after administering the first oral dose, discharging the patient;
e) alternatively, if the QTc is <500 msec at 12 hours after the first oral dose, administering a second oral dose of sotalol based on the highest target oral dose selected for step a), provided that if the patient has a creatinine clearance of ≤60 mL/min then the timing of the second oral dose is delayed as described in Table A; and,
f) discharging the patient after the second oral dose, the timing of which is described in Table A.

It is noted that if the patient has a creatinine clearance of ≤60 mL/min then additional QTc (and optionally HR and BP) measurements are typically taken to ensure the patient's QTc (and other optional measurements) remain in an acceptable range.

Typically, an acceptable range for QTc is <500 msec. Since HR and BP are patient dependent, acceptable ranges for HR and BP are determined by the medical practitioner(s) responsible for the patient.

In another aspect, persistence of QTc prolongation >500 msec means the loading is stopped and an alternative therapy sought.

In another aspect, if the patient exhibits excessive QTc prolongation (>500 msec) during IV administration, the IV infusion rate is slowed. If the patient's QTc remains excessive after slowing, then the IV is stopped. Alternative therapy can then be sought. Slowing the infusion rate means that the dose intended to be given over 1 hour is instead given over a longer period of time. Examples of this longer period include (a) 1.25 h, (b) 1.5 h, (c) 1.75 h, (d) 2 h, (e) 1.25-2 h, (f) 1.5-2 h, and (g) 1.75-2 h.

In another aspect, the patient is discharged after the first oral dose if the patient's QTc (e.g., <500 msec) (and optionally HR, and BP) are within an acceptable range.

In another aspect, the patient is discharged 4 hours after the first oral dose if the patient's QTc (e.g., <500 msec) (and optionally HR, and BP) are within an acceptable range.

In another aspect, the patient is discharged 8 hours after the first oral dose if the patient's QTc (e.g., <500 msec) (and optionally HR, and BP) are within an acceptable range.

In another aspect, the patient is discharged after the second oral dose if the patient's QTc (e.g., <500 msec) (and optionally HR, and BP) are within an acceptable range.

In another aspect, the patient is discharged 4 hours after the second oral dose if the patient's QTc (e.g., <500 msec) (and optionally HR, and BP) are within an acceptable range.

In another aspect, the patient is discharged 8 hours after the second oral dose if the patient's QTc (e.g., <500 msec) (and optionally HR, and BP) are within an acceptable range.

In another aspect, the second oral dose is administered 4, 5, 6, 7, 8, 9, 10, 11, or 12 hours after the first oral dosage (see Table A), provided that if the patient has a creatinine clearance of ≤60 mL/min then the timing of the second oral dose is delayed as described in Table A;

In another aspect, the patient is discharged once the second oral dose is administered. Examples include 9, 10, 11, 12, 13, 14, 15, 16, or 17 hours after initiation of the IV dose (see Table A), provided that if the patient has a creatinine clearance of ≤60 mL/min then these times are delayed as described in Table A. Discharge times are calculated by adding the IV time (1 hour)+delay to first oral dose (4 hours)+delay to second oral dose (4-12 hours).

In another aspect, the patient is discharged 1, 2, 3, 4, 5, 6, 7, 8, 9, 10, 11, or 12 hours after the second oral dose is taken. This delay allows for the QTc to be measured (e.g., 4-8 hours after administration).

In another aspect, the patient is discharged 9 hours after initiation of the IV dose, having received 1 oral dose of sotalol.

In another aspect, the patient is discharged 13 hours after initiation of the IV dose, having received 1 oral dose of sotalol. In another aspect, the QTc of this patient is measured 4 hours after receiving the first oral dose.

In another aspect, the patient is discharged 17 hours after initiation of the IV dose, having received 2 oral doses of sotalol. In another aspect, the QTc of this patient is measured 4 and 8 hours after receiving the first oral dose.

In another aspect, the patient is discharged 21-23 hours after initiation of the IV dose, having received 2 oral doses of sotalol. In another aspect, the QTc of this patient is measured 4-6 hours after receiving the second oral dose.

Examples of QTc interval measurements include (a) every 15 minutes (e.g., during the IV infusion), (b) 2 hours post first oral dose, (c) 4 hours post first oral dose, (d) 8 hours post first oral dose, (e) one or more of 2, 4, and 8 hours post first oral dose, (f) 2 hours post second oral dose, (g) 4 hours post second oral dose, (h) 6 hours post second oral dose, (i) 8 hours post second oral dose, (j) 12 hours post second oral dose, (k) 4-6 hours post second oral dose, (l) 4-8 hours post second oral dose, (m) one or more of 2, 4, 6, 8, and 12 hours post second oral dose, and (n) combinations thereof. Measurements at additional or other times within the 2-, 4-, 6-, 8-, and 12-hour windows can be selected. Examples include 1, 1.5, 2, 2.5, 3, 3.5, 4, 4.5, 5, 5.5, 6, 6.5, 7, 7.5, 8, 8.5, 9, 9.5, 10, 10.5, 11, 11.5, and/or 12 h.

In another aspect, when the patient has exhibited excessive QTc prolongation (>500 msec) during the IV, the first oral dose is reduced downward from the highest target oral dose selected for step a) and the renal clearance of the patient. For example, an infusion targeting 120 mg is followed by oral dosing of 80 mg or an infusion targeting 160 mg would be followed by oral dosing of 120 mg. If the patient's QTc remains excessive with the reduced dose, then oral sotalol is stopped. Alternative therapy can then be sought.

In another aspect, when the patient has exhibited excessive QTc prolongation (>500 msec) after the first oral dose, the second oral dose is reduced downward from the highest target oral dose selected for step a) considering the renal clearance of the patient. For example, a first oral dose 120 mg is followed by a second oral dose of 80 mg or first oral dose of 160 mg is followed by a second oral dose of 120 mg. If the patient's QTc remains excessive with the reduced dose, then oral sotalol is stopped. Alternative therapy can then be sought.

In another aspect, after the second oral dose, if the QTc is less than 500 msec then the patient can be discharged home on the selected oral dose of 120 or 160 mg or 80 or 120 mg (if the dose was reduced), which is to be administered orally twice daily (bid).

In another aspect, 4 to 8 hours after the second oral dose, if the QTc is less than 500 msec then the patient can be discharged home on the selected oral dose of 120 or 160 mg or 80 or 120 mg (if the dose was reduced), which is to be administered orally twice daily (bid).

In another aspect, the patient is discharged 21-25 hours after initiation of the IV dose, having received 2 oral doses of sotalol (the second being lower than the first). In another aspect, the QTc of this patient is measured 4 hours after receiving the second (reduced) oral dose. In another aspect, the QTc of this patient is measured 4 and 8 hours after receiving the second (reduced) oral dose.

In another aspect, the method further comprises: administering a third oral dose based on the highest target oral dose selected for step a) (e.g., 120 mg or 160 mg). The timing of the third oral dose is provided in Table A (see Next Oral Dosing Interval).

In another aspect, the method further comprises: administering a third oral dose that is a reduced dose based on the highest target oral dose selected for step a) (e.g., 80 mg if originally targeted 120 mg or 120 mg if originally targeted 160 mg). The timing of the third oral dose is provided in Table A (see Next Oral Dosing Interval).

In another aspect, the patient is discharged 1, 2, 3, 4, 5, 6, 7, 8, 9, 10, 11, or 12 hours after the third oral dose is taken. This delay allows for the QTc to be measured (e.g., one or both of 4 and 8 hours after administration).

In another aspect, an example of overall method for the 120 mg target therapy (CrCL>90 mL/min) is shown in FIG. 2.

Other features of the aspects will become apparent in the course of the following descriptions of exemplary embodiments that are given for illustration of the aspects and are not intended to be limiting thereof.

### EXAMPLES

### Example 1

A 76-year-old male presents with rapid highly symptomatic AF and is cardioverted in the emergency department. Prevention of recurrence of the AF is to be attempted by oral sotalol therapy. The patient is transferred to telemetry where his ECG can be continuously monitored. His creatinine clearance is determined as 91 ml/min and his weight is 97 kg. The target oral dose is selected as 160 mg orally twice daily. Baseline HR is 86 bpm, BP 130/86 and QTc 437 msec. IV sotalol is infused over 1 hour at a dose of 120 mg with HR, BP and QTc measured every 15 minutes. Four hours after the infusion is complete the QTc is measured at 460 msec and at that time 160 mg of sotalol is administered orally to the patient. Twelve hours later the patient's QTc is 450 msec and the patient is administered a second dose of sotalol orally (160 mg) and directed to take sotalol 160 mg every 12 hours. After the second oral dose (17 hours with monitoring) the patient is discharged.

### Example 2

A 62-year-old diabetic female patient has repeated runs of rapid AF that is symptomatic. She is currently in sinus rhythm and admitted to the hospital to initiate sotalol therapy. The patient weighs 68 kg and her creatinine clearance is 59 mL/min. Her target oral dose of sotalol is initially 120 mg twice daily. An intravenous line is inserted and the patient receives an infusion of 112.5 mg of sotalol IV over 1 hour. Her initial QTc was 442 msec. HR, BP and QTc is measured every 15 minutes during the infusion. Four hours after the infusion her QTc is 510 msec. It is decided that the patient should receive a lower maintenance dose of sotalol (80 mg). At 6 hours after the completion of the infusion the patient is given 80 mg sotalol orally. Given her reduced creatinine clearance she is to receive her next oral dose in 24 hours. Her QTc at that time (24 hours) is 460 msec. She receives the next oral dose of 80 mg sotalol 24 hours after the first oral dose and she is discharged on 80 mg sotalol once daily.

Numerous modifications and variations of the aspects described herein are possible in light of the above teachings. It is therefore to be understood that within the scope of the appended claims, the aspects may be practiced otherwise than as specifically described herein.

What is claimed:

1. A method of loading sotalol, comprising:
a) administering intravenously (IV) to a patient in need thereof, over 1 hour, a loading dose based on the calculated highest oral target dose based on weight, with the IV loading dose being adjusted for renal function as shown in Table A;
TABLE A

| | | | | |
|---|---|---|---|---|
| | | Minimum delay | Next oral | Discharge |
| | 1 hour IV | to first | dosing | (hours |
| CrCL | loading | oral dose | interval | after IV |
| (mL/min) | dose (mg) | (hours) | (hours) | start) |

Target Dose 120 mg, Patient Weight ≤90 kg

| | | | | |
|---|---|---|---|---|
| >90 | 90 | 4 | 12 | 9-12 h (1 oral dose), |
| | | | | 12-23 h (2 oral doses) |
| >60-90 | 125 | 4 | 12 | 9-12 h (1 oral dose), |
| | | | | 12-23 h (2 oral doses) |
| >30-60 | 112.5 | 6 | 24 | 31-39 |
| 10-30 | 112.5 | 12 | 48 | 61-69 |

Target Dose 160 mg, Patient Weight >90 kg

| | | | | |
|---|---|---|---|---|
| >90 | 120 | 4-8 | 12 | 9-12 h (1 oral dose), |
| | | | | 12-23 h (2 oral doses) |
| >60-90 | 165 | 4-8 | 12 | 9-12 h (1 oral dose), |
| | | | | 12-23 h (2 oral doses) |
| >30-60 | 150 | 6 | 24 | 31-39 |
| 10-30 | 150 | 12 | 48 | 61-69 |

b) optionally, slowing the infusion rate if the QTc increases to >500 msec and/or stopping the infusion if there is persistent QTc prolongation >500 msec;
c) if the QTc is <500 msec at 4 hours after completion of the IV loading dose, administering a first oral dose of sotalol based on the highest target oral dose selected for step a), provided that if the patient has a creatinine clearance of ≤60 mL/min then the timing of the first oral dose is delayed as described in Table A:
d) if the QTc is <500 msec at 4 hours after administering the first oral dose, discharging the patient;
e) alternatively, if the QTc is <500 msec at 12 hours the first oral dose, administering a second oral dose of sotalol based on the highest target oral dose selected for step a), provided that if the patient has a creatinine clearance of ≤60 mL/min then the timing of the second oral dose is delayed as described in Table A; and,
f) discharging the patient after the second oral dose, the timing of which is described in Table A.

2. The method of claim 1, further comprising: in step a), measuring the QTc interval every 15 minutes.

3. The method of claim 1, further comprising: measuring the QTc at one or more of 2 and 4 hours after completion of the IV.

4. The method of claim 1, further comprising: measuring the QTc at one or more of 2, 4, and 8 hours after administering the first oral dose.

5. The method of claim 1, further comprising: measuring the QTc at one or more of 2, 4, 6, 8, and 12 hours after administering the second oral dose.

6. The method of claim 1, wherein the patient's QTc increases to >500 msec and the infusion rate is slowed.

7. The method of claim 6, wherein the QTc>500 msec remains after slowing the infusion rate and the infusion is stopped.

8. The method of claim 6, wherein the infusion rate is slowed to 1.25-2 h.

9. The method of claim 1, wherein the second oral dose is administered 12 hours after the first oral dosage, provided that if the patient has a creatinine clearance of ≤60 mL/min then the timing of the second oral dose is delayed as described in Table A.

10. The method of claim 1, wherein the patient is discharged 9-17 hours after initiation of the IV dose, provided that if the patient has a creatinine clearance of ≤60 mL/min then these times are delayed as described in Table A.

11. The method of claim 1, wherein the patient is discharged 9 hours after initiation of the IV dose, having received 1 oral dose of sotalol.

12. The method of claim 1, wherein the patient is discharged 13 hours after initiation of the IV dose, having received 1 oral dose of sotalol.

13. The method of claim 1, wherein the patient is discharged 17 hours after initiation of the IV dose, having received 2 oral doses of sotalol.

14. The method of claim 1, wherein the patient is discharged 21-23 hours after initiation of the IV dose, having received 2 oral doses of sotalol.

15. The method of claim 1, wherein the patient has exhibited excessive QTc prolongation (>500 msec) during the IV and the first oral dose is reduced to 80 mg if 120 was originally targeted or 120 mg if 160 mg was originally targeted.

16. The method of claim 15, wherein oral sotalol is stopped if the patient's QTc remains excessive with the reduced dose.

17. The method of claim 1, wherein the patient has exhibited excessive QTc prolongation (>500 msec) after the first oral dose and the second oral dose is reduced to 80 mg if 120 was originally targeted or 120 mg if 160 mg was originally targeted.

18. The method of claim 17, wherein oral sotalol is stopped if the patient's QTc remains excessive with the reduced dose.

19. The method of claim 17, wherein the patient is discharged 21-25 hours after initiation of the IV dose, having received 2 oral doses of sotalol.

20. The method of claim 1, wherein the discharged patient is prescribed oral sotalol twice daily (BID) at the same dose as the second oral dose.
